(19) United States
(12) Patent Application Publication
(10) Pub. No.: US 2024/0310346 A1
(43) Pub. Date: Sep. 19, 2024

(54) Systems and Methods for Portable Gas Detector Configuration Identification

(71) Applicant: MSA Technology, LLC, Cranberry Township, PA (US)

(72) Inventors: Daniel Miller, Cranberry Township, PA (US); Jason Daschbach, Mars, PA (US); Victor Lejeune, Devizes (GB)

(21) Appl. No.: 18/582,901

(22) Filed: Feb. 21, 2024

Related U.S. Application Data
(60) Provisional application No. 63/490,036, filed on Mar. 14, 2023.

(51) Int. Cl. G01N 33/00

(52) U.S. Cl. CPC G01N 33/0075; G01N 33/0063

(57) ABSTRACT

Systems and methods are provided for a portable gas detector having an indicator that is configured to display an indication, such as a color indication, to a user of the portable gas detector, wherein the indication represents a particular gas sensor configuration setting. The portable gas detector may have one or more gas sensors configured to detect multiple species of gas and a controller configured to control configuration settings of the one or more gas sensors. The indication provided to the user allows for the efficient selection of the correct portable gas detector for each individual task, without requiring a user to rely on potentially inaccurate physical labels or to manually check device settings.

### CROSS-REFERENCE TO RELATED APPLICATIONS

This application claims priority to U.S. Provisional Application No. 63/490,036, filed Mar. 14, 2023, which is incorporated herein by reference in its entirety.

### BACKGROUND

Portable gas detectors are commonly used in a variety of settings, including by emergency service providers and by employees at various sites within the oil and gas industry. Such portable gas detectors can be used to measure either a single gas species, or multiple gas species, and often exist in a compact form that can remain with a user while performing a specific task (e.g., worksite inspection).

Because different tasks may require tailored sensor and device settings, portable gas detector sensor settings are often configured by a user. Commonly adjusted device configuration settings include the alarm limit settings and, for multi-sensor devices, which sensors are active and which gas species are actively being monitored. The ability to quickly and correctly identify which detectors have the appropriate sensors/configurations for a given task is beneficial for both efficiency and safety of the user.

### SUMMARY

Systems and methods are provided for portable gas detectors and methods related thereto that include configuration state indicators that can quickly provide information relating to gas configuration sensor settings of the detectors to a user, for example, using a color indication. A user can then rely on the configuration state indicator when identifying the appropriate portable gas detector for a given job or task.

In one aspect, the present disclosure provides a portable gas detector having one or more gas sensors configured to detect multiple species of gas, a controller configured to control configuration settings of the one or more gas sensors, and an indicator configured to display an indication to a user of the portable gas detector, wherein the indication represents a gas sensor configuration setting.

In another aspect, the present disclosure provides a method for communicating a configuration setting of a portable gas detector to a user. The method includes displaying, using an indicator, an indication to a user of a portable gas detector, wherein the indication represents a configuration setting of one or more gas sensors of the portable gas detector.

In yet another aspect, the present disclosure provides a method for controlling the use of a portable gas detector. The method includes monitoring a location of a portable gas detector, determining whether the sensor configuration settings of the detector are permitted for the location of the detector based on preset location allowances, and displaying an alert signal to a user of a portable gas detector if the sensor configuration settings are not permitted for the location of the detector.

### BRIEF DESCRIPTION OF DRAWINGS

FIG. 1 is a diagram illustrating a portable gas detector, in accordance with one aspect of the present disclosure.

FIG. 2 is a diagram illustrating multiple portable gas detectors positioned within a multi-unit charger, in accordance with one aspect of the present disclosure.

FIG. 3 is a diagram illustrating multiple portable gas detectors positioned within a multi-unit charger and an associated communications device, in accordance with one aspect of the present disclosure.

FIG. 4 is a diagram illustrating a portable gas detector and a radio-frequency identification tag, in accordance with one aspect of the present disclosure.

FIG. 5 is a diagram illustrating a window of a configuration ID color selection program on a remote device, in accordance with one aspect of the present disclosure.

FIG. 6 is a flowchart illustrating a method for communicating a configuration setting of a portable gas detector to a user.

FIG. 7 is a flowchart illustrating a method for controlling the use of a portable gas detector.

FIG. 8 is a diagram illustrating a portable gas detector positioned within a specific area surrounding a worksite and outside of a specific area surrounding a building, in accordance with one aspect of the present disclosure.

FIG. 9 is a diagram illustrating a portable gas detector in a location-based warning state, in accordance with one aspect of the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer-readable medium and executed by a computer or processor, whether such computer or processor is explicitly shown. While each of the figures illustrates a particular embodiment for purposes of illustrating a clear example, other embodiments may omit, add to, reorder, and/or modify any of the elements shown in the figures.

### DETAILED DESCRIPTION

In the following detailed description of the embodiments of the disclosure, reference is made to the accompanying drawings that form a part hereof, and in which are shown by way of illustration specific embodiments in which the disclosure may be practiced. It should be understood, however, that it is not intended to limit the disclosure to the forms disclosed, but on the contrary, the disclosure is to cover all modifications, equivalents, and alternatives falling within the spirit and the scope of the disclosure. It is to be understood that other embodiments may be utilized and that changes may be made without departing from the scope of the present disclosure. The following description is, therefore, not to be taken in a limiting sense.

Often times users of portable gas detectors have multiple detectors configured in different ways for different purposes. To promote accessibility, these gas detectors are often stored in close proximity (e.g., on a common equipment rack). With several detectors, each in a potentially different configuration, quickly selecting the correct gas detector(s) when preparing to perform a particular task can be difficult, especially when each device shares a similar or, in some cases, identical outward appearance. In some instances, a user may be required to turn on each gas detector one by one in order to manually check the gas sensor configuration settings, including which sensors are installed in each device. Given that portable gas sensors often have lengthy start up times, valuable time can be wasted during this process. This lost time can lead to adverse outcomes, especially in emergency situations. Furthermore, a user may attempt to quickly choose a device without manually checking the settings, increasing the potential for a safety issue if a gas detector with the wrong configurations is selected.

One mechanism to mitigate these issues could utilize permanent or semi-permanent physical alterations to try to distinguish gas detectors. For instance, different external housing identifiers could be used to try to assist a user in easily differentiating various gas detectors. Similarly, stickers, tags, or labels could be applied to detectors. However, these mechanical solutions may come with several drawbacks. For example, stickers and tags could fall off or fade in UV light over time, labels could be smeared and become illegible, and different colored housings could limit the number of configurations that can be discerned. Furthermore, when the configuration settings of a gas detector are changed, the sticker, tag, or label would no longer be accurate unless it is also changed or replaced. Thus, these solutions could run the risk of potentially misinforming a user selecting a gas detector(s) for a given situation. For instance, one may select a gas detector based on a sticker label indicating that it has certain sensor configurations, when in reality, the device has had its sensor configurations changed since the creation of the label. This potential source of confusion could present a serious safety risk to any user selecting a gas detector(s) for a given application.

Embodiments of systems and methods herein may provide portable gas detectors and methods related thereto that include configuration state indicators that can provide information relating to gas configuration sensor settings of the detectors to a user, for example, using a color indication. The indication provided to the user can thereby allow them to efficiently select the appropriate portable gas detector for the task at hand, without relying on potentially inaccurate physical labels or having to check the setting of each device manually.

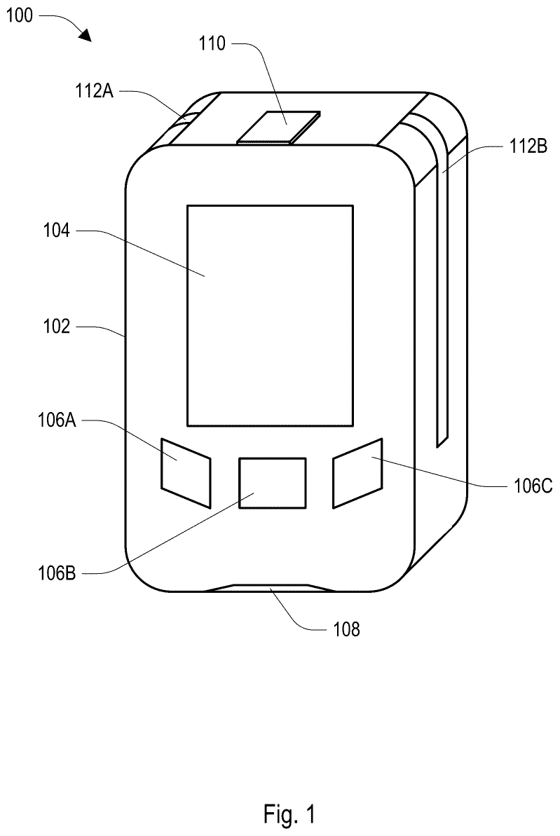

FIG. 1 depicts a portable gas detector 100 which is specifically configured to allow a user to quickly identify its gas sensor configuration settings. The gas sensor includes a housing 102, a primary display 104, several input buttons 106A, 106B, 106C, a charging port 108, an operational state indicator 110, and two configuration state indicators 112A, 112B positioned on either side of the housing 102. The indicators 112A, 112B may be configured to display an indication to a user, such as a visible color indication, wherein the indication represents a particular gas sensor configuration setting of the portable gas detector 100. Therefore, based on the indication, a user of the portable gas detector 100 may quickly determine which applications the portable gas detector is appropriate for use in. In one example, the indication may be provided regardless of whether the gas detector 100 is in a powered or unpowered state.

Contained within the housing 102, the portable gas detector 100 may include one or more gas sensors (not depicted) configured to detect multiple species of gas. For instance, the portable gas detector 100 may include gas sensors configured to detect oxygen, carbon monoxide, hydrogen sulfide, sulfur dioxide, and carbon dioxide levels, as well as lower explosive limit (LEL) sensors and photoionization detectors (PID), among others. As discussed, various applications may require several of these sensors to be housed within the portable gas detector 100 at specific configuration settings. The gas sensor configuration settings can include alarm limit settings (e.g., 20% LEL) for the various species of gas capable of being detected by the portable gas detector 100. The gas sensor configuration settings may additionally or alternatively include configuration settings controlling which of the gas sensors within the portable gas detector 100 are currently activated and, relatedly, which species of gas are being actively detected.

Also contained within the housing 102, the portable gas detector 100 may include a controller (not depicted) configured to control the configuration settings of the one or more gas sensors. The controller may be in communication with various electrical components within the portable gas detector as well as the primary display 104, several input buttons 106A, 106B, 106C, the charging port 108, the operational state indicator 110, and two configuration state indicators 112A, 112B. Among other functionality, the controller may be configured to cause the indicators 112A, 112B to display the indication at a specific time (e.g., when charging).

The configuration state indicators 112A, 112B may be configured to simultaneously display an indication to a user of the portable gas detector 100 representing a particular gas sensor configuration setting. The indication may specifically represent alarm limit settings, which of the one or more gas sensors are active, and/or which gas species are being actively detected. In order to remain easily distinguishable when placed within a bin or similar container, the indicators 112A, 112B may be configured to display the indication on at least two geometric sides, at least three geometric sides, or from any unobstructed viewing angle of the portable gas detector 100. For instance, as shown in FIG. 1, the indicators 112A, 112B may be positioned to be visible from the left, right, and top side of the portable gas detector 100, where the top side is the side opposite of the side where the charging port 108 is located. The indicators 112A, 112B being configured to display the indication on the side that is opposite of the charging port 108 may ensure that the any indication provided to a user is not obstructed by a charging apparatus when the portable gas detector 100 is being charged, whether by a multi-unit charger or a single charger. Although two indicators 112A, 112B are depicted in FIG. 1, it should be appreciated that alternative arrangements involving only a single indicator or additional indicators may alternatively be used.

The indicators 112A, 112B may be configured to display substantially all of the color spectrum. For instance, the indicators 112A, 112B may include one or more red green blue light emitting diodes (RGB LEDs) capable of emitting a variety of color variations as the color indication. This can allow for a large number of gas sensor configurations to be associated with a specific color indicator, since there are a large number of color variations distinguishable to the human eye. For example, different shades of blue may represent different gas species limits within the same gas sensor activation settings (e.g., 10% LEL [light blue], 20% LEL [navy blue]).

The color indication representative of each individual gas configuration setting may be specifically selected by a user of the portable gas detector. For instance, the color indication may be set within the internal settings of the device as accessed within a menu of the primary display 104. Alternatively, a user may select color indications associated with various gas sensor configurations using cloud platform software on a remote network device, which would then apply the selected color settings to all portable gas detectors present on the network. When selecting the color indication, the portable gas detector or remote network device may provide guidance, recommending certain color indications for specific gas sensor configurations through prompts on the device or on the cloud platform software.

Rather than relying on a user to provide the color indication settings, the controller of the portable gas detector may be configured to automatically determine a color indication to be displayed based on determined gas sensor configurations of the portable gas detector. For instance the controller may compare the sensor configuration settings to a number of known sensor configuration setting ranges, wherein each sensor setting range corresponds to a particular color indication, and then determine the proper color indication to be displayed. In this manner, the portable gas detector 100 may provide smart color indication selection for common device configurations where no action would be required by the user. Such automatic determination of the color indication can maintain consistent configuration setting identification, and help avoid the situation where one user is forced to decipher the custom color indication settings of another user.

The indicators 112A, 112B may be configured to display the indication at specific times or when the portable gas detector 100 is in a specific state. For example, the indicators 112A, 112B may be configured to display the indication when the portable gas detector 100 is in a power-off state, which can allow a user to select the correct portable gas detector for the job without having to wait for the device to power on. Conversely, in order to avoid power consumption, the indicators 112A, 112B may be configured to display the indication when the portable gas detector 100 enters a wake-up state, such as when a user pushes one of the input buttons 106A, 106B, 106C or shakes the portable gas detector 100. Pushing a single input button may instead cause the indication to be displayed without the device entering a full wakeup state. Similarly, indicators 112A, 112B may be configured to display the indication when the portable gas detector 100 is in a charging state.

In order to avoid confusion, the information provided to a user of the portable gas detector 100 by the indicators 112A, 112B may be distinct from that provided by the primary display 104 and the operational state indicator 110. Specifically, the indicators 112A, 112B may be limited to only displaying an indication representative of the gas sensor configurations. As described herein, providing an indication representative of the gas sensor configurations using an indicator that does not also provide additional information to the user can provide useful functionality and help to avoid confusion that could lead to a potential safety risk. The operational state indicator 110 may be configured to provide one or more operational color indications corresponding to an operational state of the gas detector. Example operational states that can be associated with an operational color indication include a charging state (e.g., red), power-off state (e.g., no color indication), power-on state (e.g., green), or a calibration state (e.g., yellow). The indicators 112A, 112B may be limited to providing color indications that are separate from the operational color indications in order to avoid confusion. Alternatively, the different positions of the indicators 112A, 112B and the operational state indicator 110 on the device may be sufficient alone to avoid confusion without the need to limit the available colors for the color indication provided by the indicators 112A, 112B.

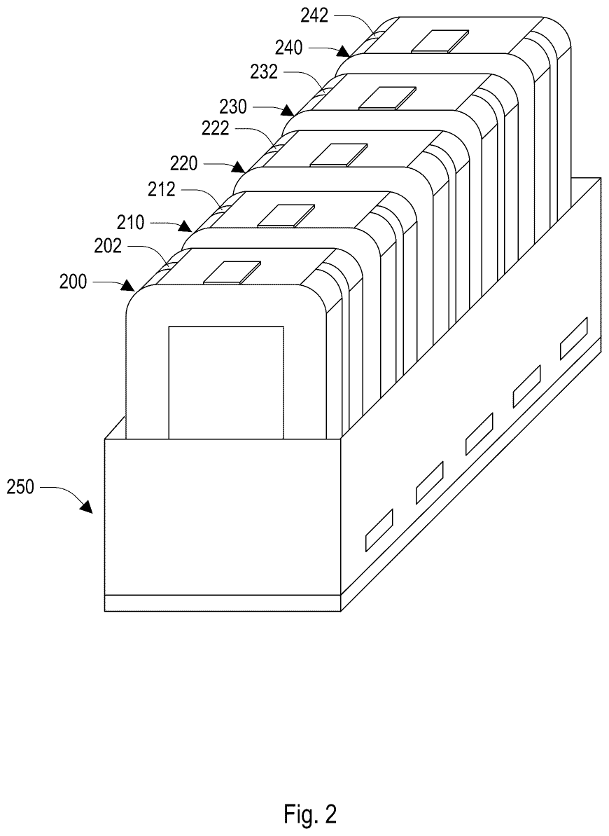

FIG. 2 depicts numerous, physically-indistinguishable portable gas detectors 200, 210, 220, 230, 240 positioned within a multi-unit charger 250. Each of the portable gas detectors 200, 210, 220, 230, 240 includes a specific color indication 202, 212, 222, 232, 242 representative of the specific gas sensor configuration settings of each detector. Each color indication 202, 212, 222, 232, 242 may be visible while charging occurs, as shown. In this manner, a user trying to select the appropriate portable gas detector(s) for a given job can quickly ascertain which of detectors 200, 210, 220, 230, 240 have the gas sensor configuration settings required for the job, without having to turn on, or even touch, any device.

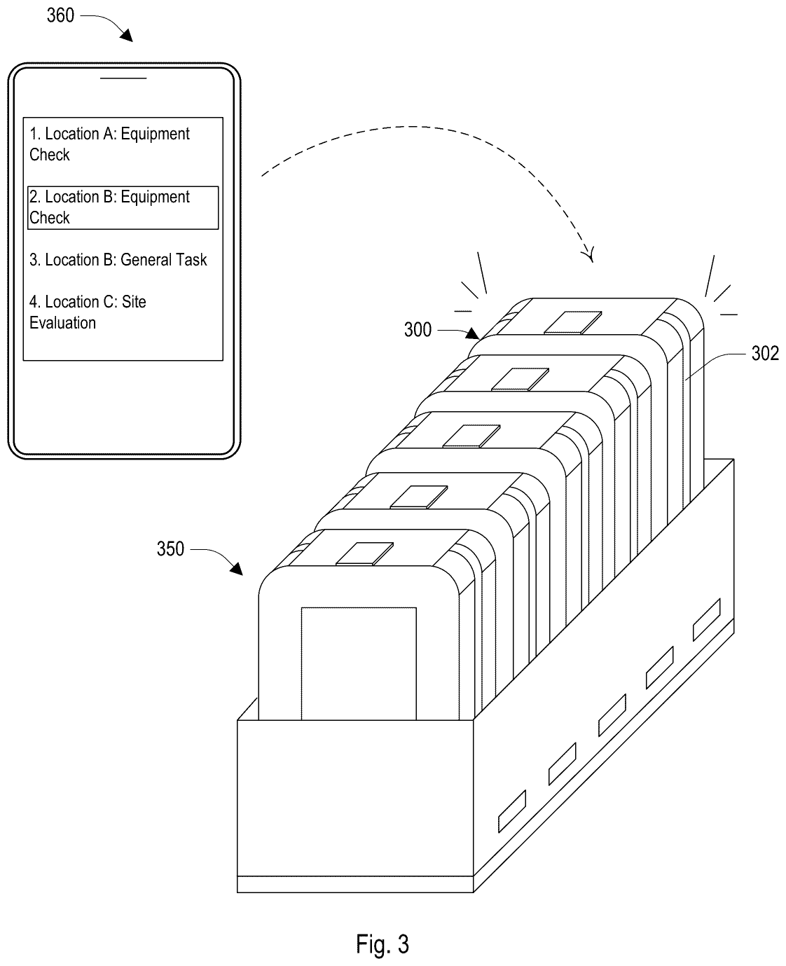

FIG. 3 depicts multiple portable gas detectors positioned within a multi-unit charger 350 and an associated network device 360 (e.g., mobile phone, computer). As shown, a user may use the remote network device 360 to select a job with known parameters or otherwise select the parameters required for said job. In this manner, the remote network device 360 may quickly inform a user of which devices are available for the job, as well as the current gas sensor configuration settings of each device. The network device 360, or another device connected to the network, may then determine which portable gas detector(s) within the network are appropriate for the job and provide an alert signal to the portable gas detector(s). After receiving the alert signal, the device 300 with the appropriate gas sensor configuration settings may be configured to provide an alert to the user, so that the user can easily distinguish the appropriate device for the job.

The alert provided by the portable gas detector 300 may be in the form of a change to the indicator 302 of the device. For instance, the indicator 302 may start providing the color indication corresponding to its gas sensor configuration settings. If the color indication was already being displayed by the indicator 302, the color indication may undergo a change in form (e.g., start to blink on and off). Furthermore, the alert provided to the user need not rely solely on the color indicator 302, and can additionally or alternatively rely on other communication aspects of the portable gas detector 300. For example, the other display components of the detector 300 may provide a visual signal, the detector 300 may vibrate, and/or the detector 300 may produce an alert sound. Based on the alert provided, the user may quickly determine which portable gas detector to select and then proceed with the job.

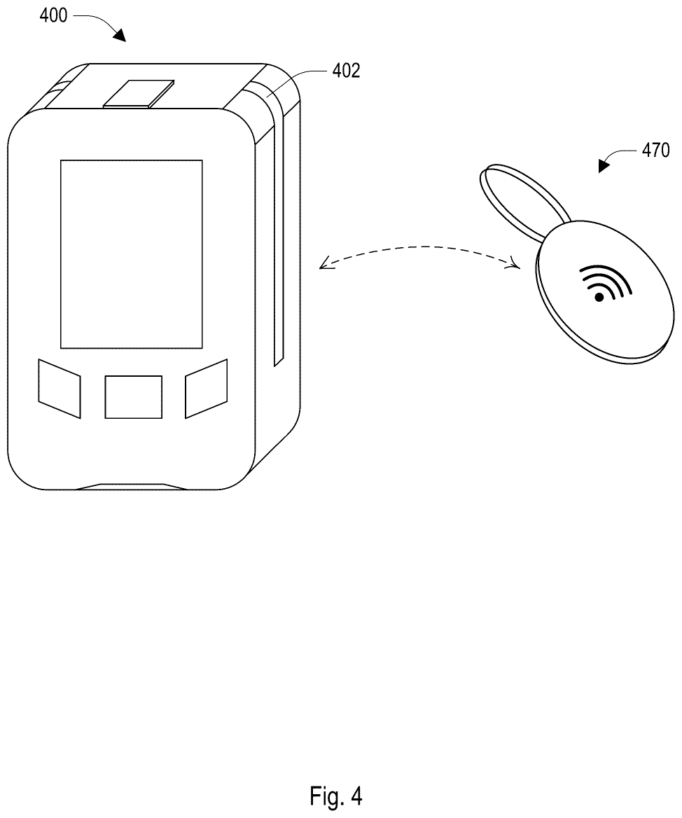

FIG. 4 depicts a portable gas detector 400 in close proximity with a radio-frequency identification (RFID) tag 470. The portable gas detector 400 may be configured so that an indicator 402 is set up to display a color indication representative of gas sensor configuration settings when the RFID tag 470 is brought within close proximity of the portable gas detector 400. The RFID tag 470 may be specifically associated with an individual user, and may be part of a broader safety plan requiring the user to tag out specific safety devices before using them. Based on the unique RFID tag sensed, the portable gas detector 400 may be configured to function in a particular manner. If the RFID tag 470 is associated with an individual user who is known within the network system to typically perform jobs that require gas sensor configuration settings different from those of the portable gas detector 400, the detector 400 may provide an alert signal to the user. For example, if a user typically performs jobs that require a sulfur dioxide sensor, and the detector 400 does not have such a sensor active, an alert message may be displayed on the device, thereby prompting the individual user to recheck whether the appropriate detector has been selected. The RFID-based alert may take alternate forms, such as a flashing of the color indication provided by the indicator 402.

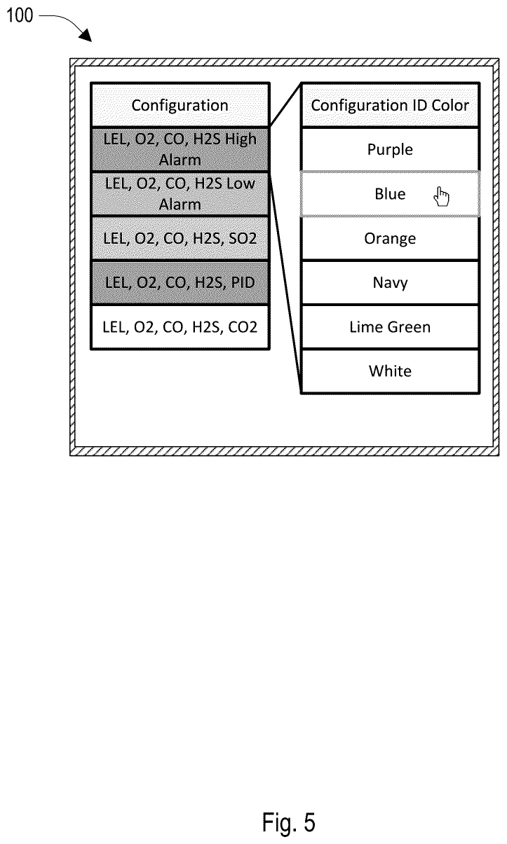

FIG. 5 depicts an example configuration ID color selection program on a remote device, such as a computer or cell phone, which can allow an individual to select the color indications associated with gas sensor configuration settings for each portable gas detector in a network. As shown, an individual may select specific colors for each setting (e.g., LEL, O₂, CO, H₂S High Alarm: Blue), and these selected indication settings can then be provided to the portable gas detectors on the network. By relying on a cloud software program in this manner, near-instantaneous dissemination of consistent color indication settings to all gas detectors on the network can be achieved.

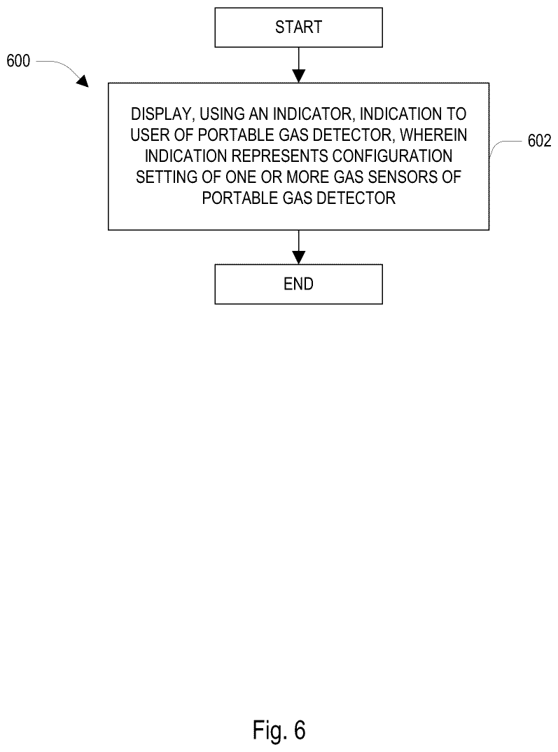

FIG. 6 depicts a method 600 for communicating a configuration setting of a portable gas detector to a user. At 602, an indication may be displayed to a user of a portable gas detector using an indicator, wherein the indication represents a configuration setting of one or more gas sensors of the portable gas detector. The configuration settings may include at least one of: an alarm limit setting for the multiple species of gas, which of the one or more gas sensors are active, or which species of gas are being actively detected. The indication may specifically be a color indication.

The method 600 may further include comparing the configuration settings to a number of known sensor setting ranges, wherein each sensor setting range corresponds to a particular indication. The process of comparing the configuration settings may be performed by the portable gas detector or another device connected thereto. For example, a controller of the portable gas detector may determine that the gas sensor configuration settings (e.g., several sensors including LEL, O₂, CO, H₂S, PID) correspond to a specific indication within the known sensor setting ranges stored on the portable gas detector. Next, the method 600 may further include determining the indication to be displayed based on the corresponding sensor setting range. As with the comparison, the process of making the determination may be performed by the portable gas detector or another device connected thereto. Rather than determining the indication automatically, the method 600 may instead prompt a user to select the indication to be displayed. The prompt may be provided to the user through, for example, a display of the portable gas detector.

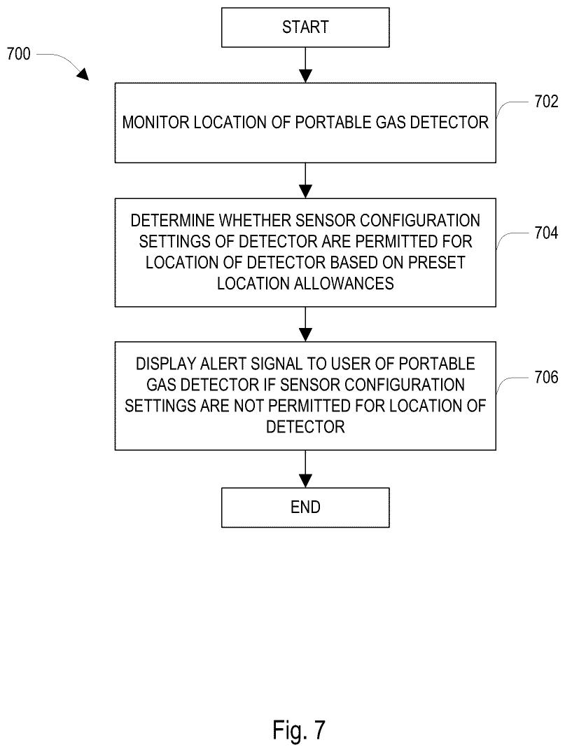

FIG. 7 depicts a method 700 for controlling the use of a portable gas detector. At 702, the location of a portable gas detector is monitored. A number of techniques may be used to determine the location of the portable gas detector, including but not limited to, global positioning system (GPS) tracking, Wi-Fi location tracking, or other geolocation techniques. At 704, it is determined whether the sensor configuration settings of the detector are permitted for the location of the detector based on preset location allowances. The process of making the determination may be performed by the portable gas detector or another device in communication with or connected thereto. For instance, a remote server on a network which the portable gas detector is connected to may receive a GPS position of the device and compare the GPS position to acceptable geographical areas for devices that have the same gas sensor configurations as the portable gas detector. At 706, an alert signal is displayed to a user of a portable gas detector if the sensor configuration settings are not permitted for the location of the detector. The alert signal may be overridable by a user of the portable gas detector. Alternatively, the method 700 may further include providing a deactivation signal to a second device if the sensor configuration settings are not permitted for the location of the detector.

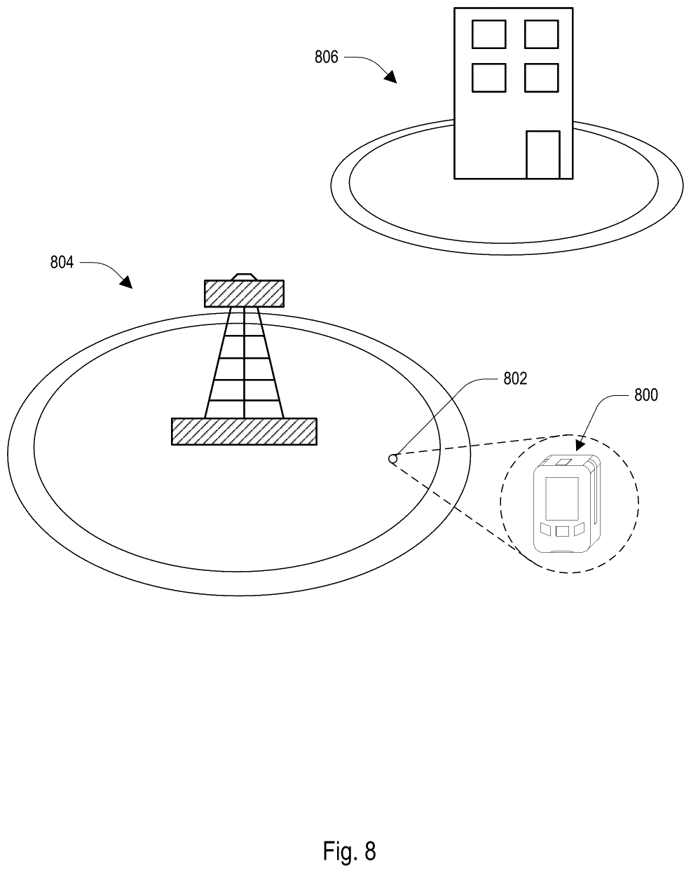

FIG. 8 depicts a portable gas detector 800 located at a first geographical location 802. As shown, the first location 802 is within a geographical area surrounding a worksite 804. In accordance with the method 700, it may be determined whether the gas sensor configuration settings of the portable gas detector 800 are suitable for the geographical area surrounding the worksite 804. If they are not suitable, an alert may be displayed to a user of the device. As can also be seen, the first location 802 is positioned outside of a geographical area surrounding a building 806. In accordance with the method 700, the portable gas detector 800 may have gas sensor configuration settings that make it only suitable to operate within the geographical area 806, and therefore may display an alert signal to the user for any location outside of the geographical area 806. In this manner, certain portable gas detectors may be limited to specific task areas based on their gas sensor configuration settings. FIG. 8 represents only a few possible geographical limitations, and it should be readily appreciated that other geographical limitations may be imposed on the portable gas detector 800.

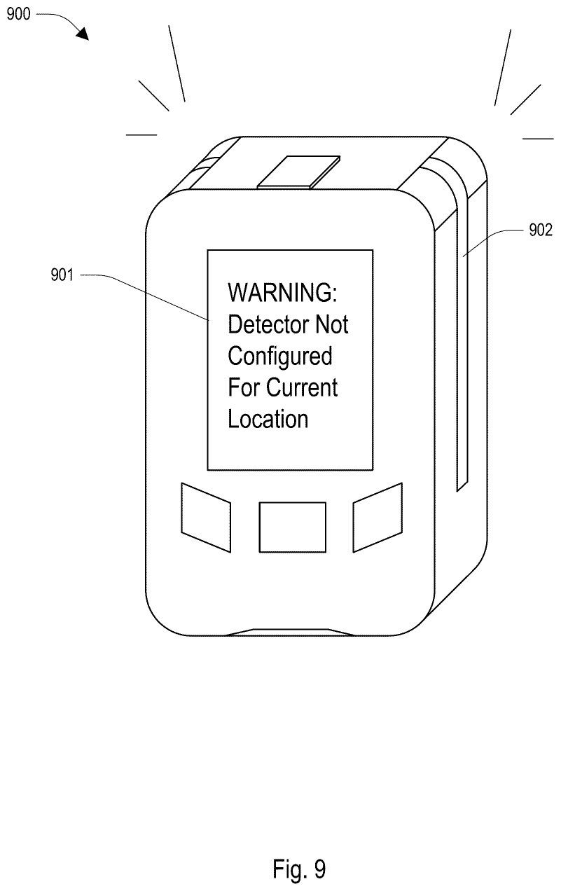

FIG. 9 depicts an example alert signal 901 being provided to a user of the portable gas detector 900 in accordance with the method 700. The example alert signal displayed states “WARNING: Detector Not Configured For Current Location.” Other warning statements, symbols, and sounds may be used to warn the user of the device. As shown, at least one indicator 902 of the device may be configured to flash or undergo a different state change in order to provide the alert signal to the user. Because the indicator 902 may be specifically configured to provide only information relating to the gas sensor configuration settings of the portable gas detector 900, this alert step may immediately inform the user that the issue with the device is related to the gas sensor configuration settings.

Although the disclosure has been described in detail for the purpose of illustration based on what are currently considered to be the most practical and preferred embodiments or aspects, it is to be understood that such detail is solely for that purpose and that the disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment or aspect can be combined with one or more features of any other embodiment or aspect.

1. A portable gas detector comprising:
one or more gas sensors configured to detect multiple species of gas;
a controller configured to control configuration settings of the one or more gas sensors;
an indicator configured to display an indication to a user of the portable gas detector, wherein the indication represents a gas sensor configuration setting.

2. The portable gas detector of claim 1, wherein the indication displayed to the user by the indicator is a color indication.

3. The portable gas detector of claim 1, wherein the gas sensor configuration setting includes an alarm limit setting for at least one species of gas, which of the one or more gas sensors are active, or which species of gas are being actively detected.

4. The portable gas detector of claim 1, wherein the indicator is configured to display the indication when the portable gas detector is in a charging state.

5. The portable gas detector of claim 1, wherein the indicator is configured to display the indication when the portable gas detector is in a power-off state.

6. The portable gas detector of claim 1, wherein the indicator is configured to display the indication when the portable gas detector enters a wake-up state.

7. The portable gas detector of claim 1, wherein the indicator is configured to display the indication if the portable gas device is physically shaken by a user.

8. The portable gas detector of claim 1, wherein the indicator is configured to display the indication if a radio-frequency identification (RFID) tag is brought within close proximity of the portable gas detector.

9. The portable gas detector of claim 1, wherein the indicator is configured to display the indication on at least two geometric sides of the portable gas detector.

10. The portable gas detector of claim 1, wherein the controller is configured to:
compare the sensor settings to a number of known sensor setting ranges, wherein each sensor setting range corresponds to a particular indication; and
determine the indication to be displayed.

11. The portable gas detector of claim 1 further comprising:
a second indicator configured to display a second indication to a user of the portable gas detector, wherein the second indication corresponds to an operational state of the portable gas detector.

12. The portable gas detector of claim 11, wherein the operational state includes a charging state, a power-off state, a power-on state, or a calibration state.

13. The portable gas detector of claim 1, wherein the indicator includes a red green blue light emitting diode (RGB LED).

14. A method for communicating a configuration setting of a portable gas detector to a user, the method comprising:
displaying, using an indicator, an indication to a user of a portable gas detector, wherein the indication represents a configuration setting of one or more gas sensors of the portable gas detector.

15. The method of claim 14, wherein the configuration settings include at least one of an alarm limit setting for at least one species of gas, which of the one or more gas sensors are active, or which species of gas are being actively detected.

16. The method of claim 14, further comprising:
comparing, using a controller, the configuration setting to a number of known sensor setting ranges, wherein each sensor setting range corresponds to a particular indication.

17. The method of claim 16, further comprising:
determining, using the controller, the indication to be displayed based on the corresponding sensor setting range.

18. The method of claim 16, further comprising:
prompting, using a display of the portable gas detector, a user to select the indication to be displayed.

19. A method for controlling the use of a portable gas detector, the method comprising:
monitoring a location of a portable gas detector;
determining whether the sensor configuration settings of the detector are permitted for the location of the detector based on preset location allowances; and
displaying an alert signal to a user of a portable gas detector if the sensor configuration settings are not permitted for the location of the detector.

20. The method of claim 19, further comprising:
providing a deactivation signal to a second device if the sensor configuration settings are not permitted for the location of the detector.
